(19) United States
(12) Patent Application Publication
(10) Pub. No.: US 2016/0008621 A1
(43) Pub. Date: Jan. 14, 2016

(54) Biological Resonance Chip

(71) Applicant: Yu-Mei Shen, New Taipei City (TW)

(72) Inventor: Yu-Mei Shen, New Taipei City (TW)

(21) Appl. No.: 14/331,206

(22) Filed: Jul. 14, 2014

(51) Int. Cl. A61N 5/02

(52) U.S. Cl. CPC A61N 5/02

(57) ABSTRACT

The present invention is a biological resonance chip utilizing a human biological micro-wave and coupling resonance technique by consisting of a printed circuit sticker at which at least a biological micro-wave receiver, a high frequency oscillating chip and at least a biological micro-wave transmitter are provided and electrically connected. By the aforesaid configuration, a product made by the printed circuit sticker can be placed at an arbitrary area of a human body from which a human biological micro-wave is emitted, picked by the biological micro-wave receiver, received by the high frequency oscillating chip for amplifying a power level and transmitted by the biological micro-wave transmitter to the human body for improving a health condition thereof. Further advantages of permanent resonance with a human body by a concentrated resonance frequency without a hazard from radiation are also achieved.

### TECHNICAL FIELD OF THE INVENTION

The present invention is to provide a health sticker with a passive resonance structure, particularly a biological resonance chip to produce a permanent resonance with a human body by a concentrated resonance frequency without a hazard from radiation.

### DESCRIPTION OF THE PRIOR ART

A health sticker of the prior art is to place a sticker on an area of a human body where a healthy improvement is desired and then emit infrared radiation from the magnet inside the health sticker for enhancing the metabolism and circulation of the user so as to improve his physical condition.

However the health sticker of the prior art has certain issues in application that the radioactive magnet is potential to inflict hazard on a human body and the radiation of the magnet will be decaying gradually after a period of application and the effectiveness of the magnet is eventually lost. Further the band of radiation frequency of the magnet is so broad that the emitted energy cannot concentrate on a required range and thus the effectiveness on health improvement is limited.

### SUMMARY OF THE INVENTION

An objective of the present invention is to place a biological resonance chip on a human body in order to pick up a human biological micro-wave emitted from a human body by a biological micro-wave receiver, to passively receive a transmission of human biological micro-wave and amplify a power thereof by a high frequency oscillating chip and to transmit the power amplified transmission of human biological micro-wave by a biological micro-wave transmitter to the human body. Thereby the disadvantages of the health stickers of the prior art, such as potential radioactive hazard to human body, decaying radiation through time and wandered energy emission because of broad radiation frequency range, are resolved through the aforesaid configuration for achieving a permanent resonance with a human body by a concentrated resonance frequency without a hazard from radiation.

In order to achieve this objective, the present invention consists of a printed circuit sticker, at least a biological micro-wave receiver, a high frequency oscillating chip and at least a biological micro-wave transmitter. The biological micro-wave receiver, the high frequency oscillating chip and the biological micro-wave transmitter are electrically connected and positioned at the printed circuit sticker. Therefore, when the resonance chip made by the printed circuit sticker is placed on an arbitrary area of a human body, the biological micro-wave receiver will pick up a human biological micro-wave emitted from a human body, a transmission of human biological micro-wave will be amplified by the high frequency oscillating chip in power level and then the power amplified transmission of human biological micro-wave transmitted by the biological micro-wave transmitter to the human body for improving the health thereof. Whereby the biological micro-wave receiver receive the human biological micro-wave in a passive manner that radioactive hazard does not exist, the resonance with human body is permanent and the human biological micro-wave is simply amplified to a concentrated frequency range. Therefore the advantages of permanent resonance with a human body by a concentrated resonance frequency without a hazard from radiation are achieved.

### BRIEF DESCRIPTION OF THE DRAWINGS

FIG. 1 is a front view illustrating a preferred embodiment of the present invention.

FIG. 2 is a side view illustrating the preferred embodiment of the present invention.

FIG. 3 is a structural block diagram illustrating the preferred embodiment of the present invention.

FIG. 4 is a schematic diagram illustrating the operation of the preferred embodiment of the present invention. FIG. 5 is a schematic diagram illustrating an application of the preferred embodiment of the present invention.

FIG. 6 is a three-dimensional diagram illustrating another preferred embodiment of the present invention.

FIG. 7 is a structural block diagram illustrating another preferred embodiment of the present invention.

FIG. 8 is a schematic diagram illustrating the operation of another preferred embodiment of the present invention.

FIG. 9 is a front view diagram illustrating the other preferred embodiment of the present invention.

FIG. 10 is a structural block diagram illustrating the other preferred embodiment of the present invention.

FIG. 11 is a schematic diagram illustrating the operation of the other preferred embodiment of the present invention.

### DETAILED DESCRIPTION OF THE PREFERRED EMBODIMENTS

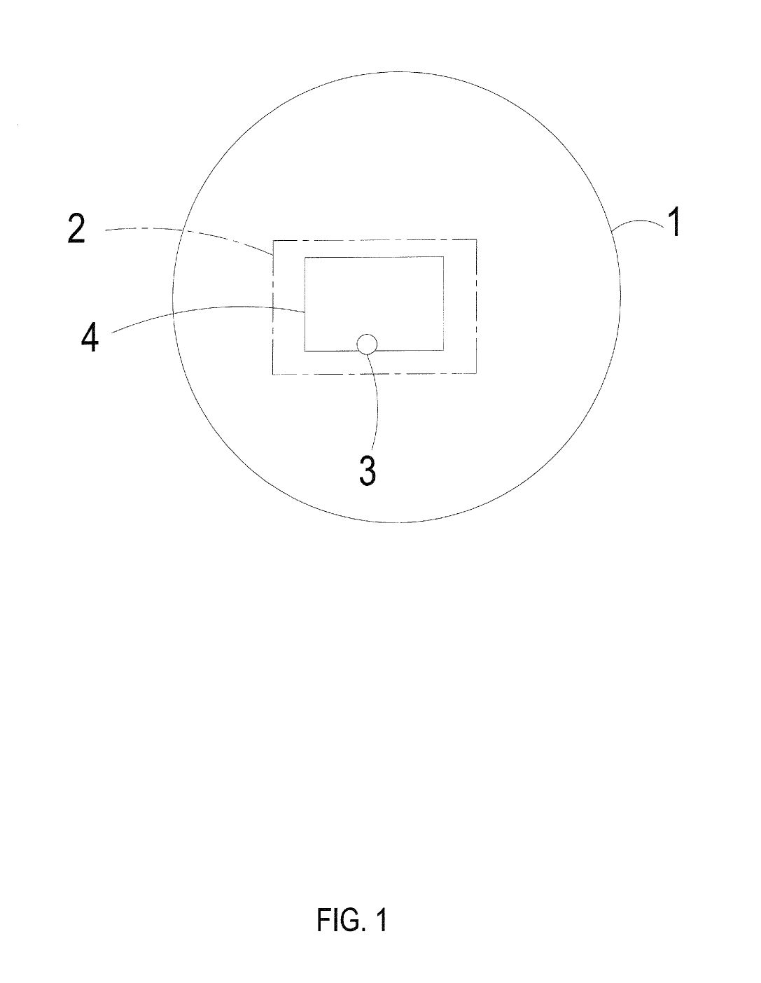

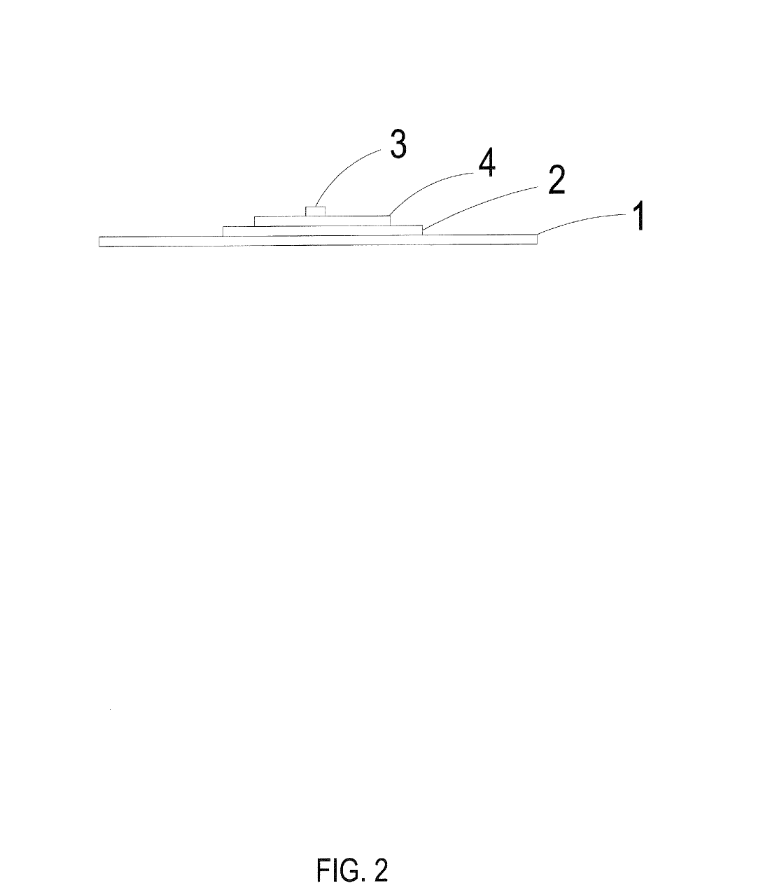

Please refer FIG. 1 to Fig, 3 illustrating a front view, side view and structural block diagrams of a preferred embodiment of the present invention that consists of

A printed circuit sticker 1, which is able to be placed on an arbitrary area of a human body;

At least a biological micro-wave receiver 2, which is positioned at the printed circuit sticker 1, for picking up a human biological micro-wave emitted from the human body;

A high frequency oscillating chip 3, which is positioned at the printed circuit sticker 1, for passively receiving a transmission of human biological micro-wave from the biological micro-wave receiver 2 and then amplifying a power level of the transmission of human biological micro-wave accordingly; and

A biological micro-wave transmitter 4, which is positioned at the printed circuit sticker 1, for receiving the power amplified transmission of human biological micro-wave from the high frequency oscillating chip 3 and then transmitting the power amplified transmission of human biological micro-wave to the human body.

Both the biological micro-wave receiver 2 and the biological micro-wave transmitter 4 are made of metal substance and the high frequency oscillating chip 3 consists of a biological signal processing circuit and a memory.

The biological micro-wave receiver 2, the high frequency oscillating chip 3 and the biological micro-wave transmitter 4 are electrically connected.

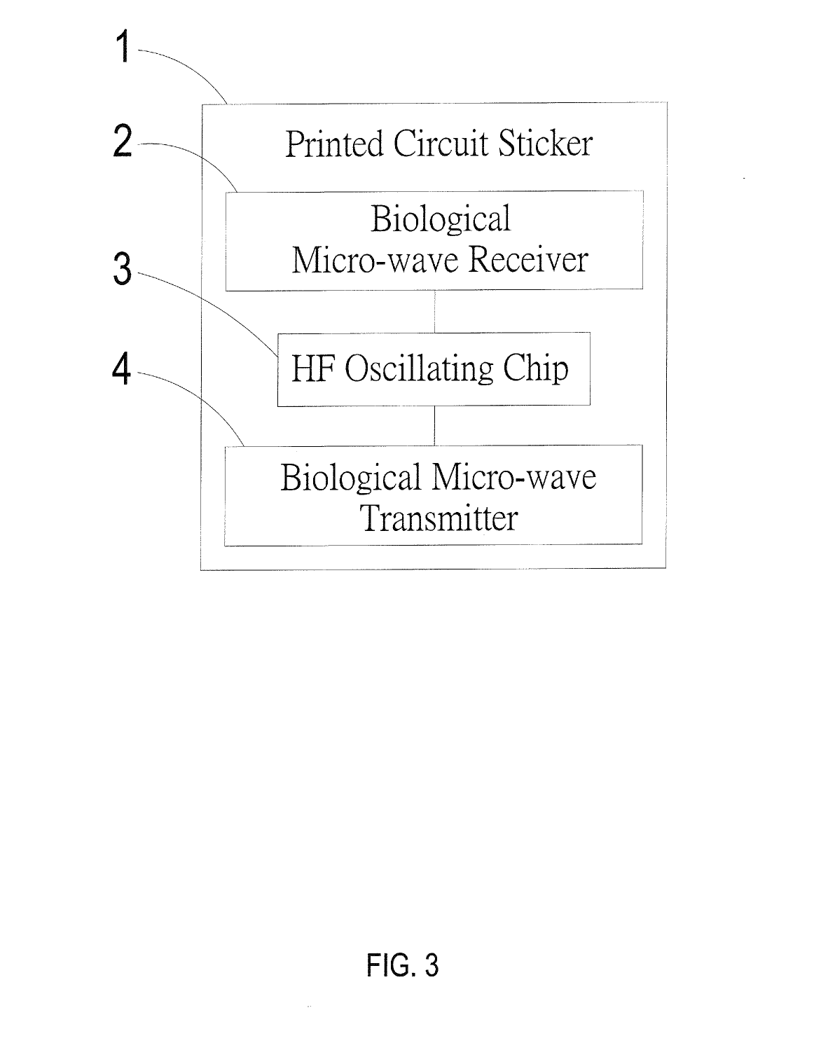

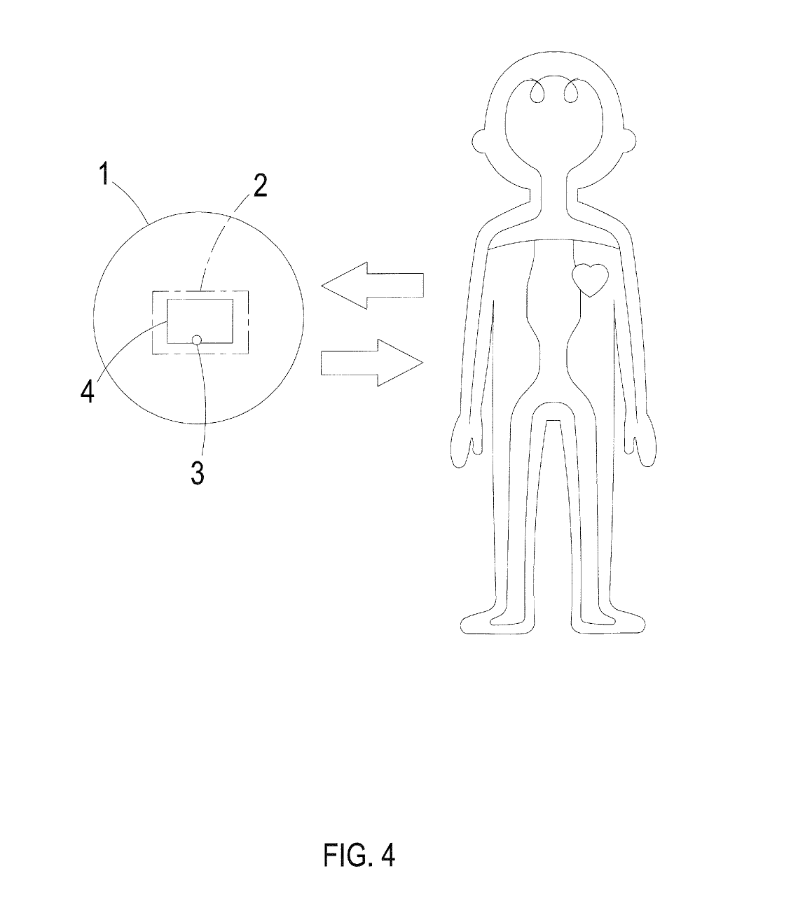

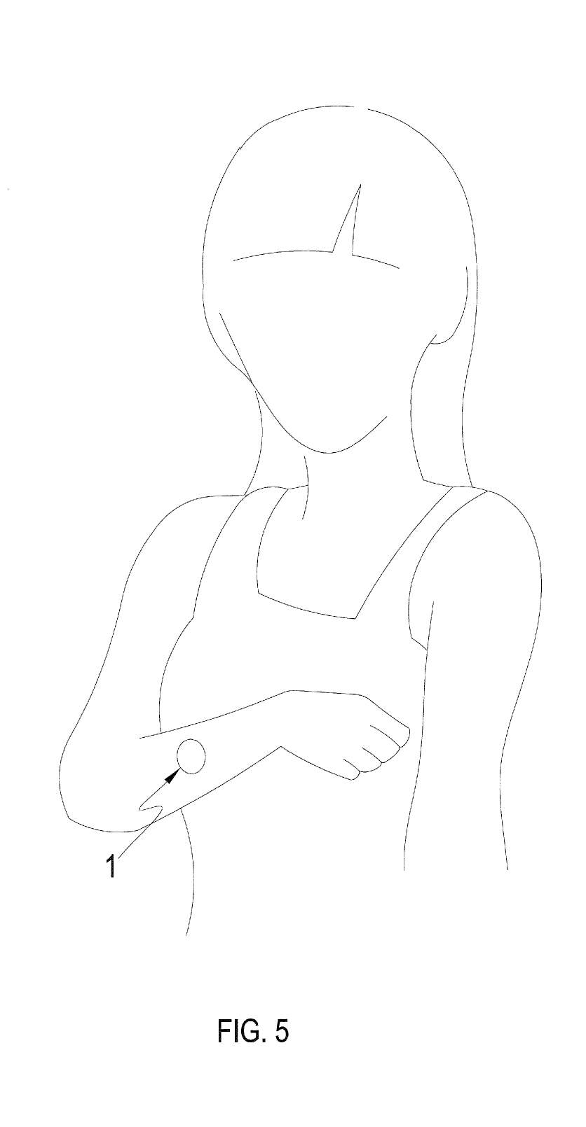

Please refer FIGS. 3 to 5 illustrating a structural block diagram and the schematic diagrams for operation and application of the preferred embodiment of the present invention. As clearly shown, this embodiment is in a sticker form that a user can place the printed circuit sticker 1 on an arbitrary area of a human body for completing the application. A human body will emit certain human biological micro-wave that will be detected and picked up by the biological micro-wave receiver 2. The human biological micro-wave signal in receipt will be transmitted through a passive resonance by a high frequency coupling resonance technique in order to amplify the power level of the human biological micro-wave in transmission. Then the power amplified transmission of the human biological micro-wave will be transmitted from the biological micro-wave transmitter 4 to the human body, which can enhance the circulation condition within the area adjacent to the printed circuit sticker 1 after receives the power amplified transmission of the human biological micro-wave so as to achieve the improvement on health (such as the enhancement on acupuncture meridians of human body, improvement on flow rate and speed of blood in a vein, and so on). After the health condition of the human body improved, the biological micro-wave thereof will gradually increase and then through the aforesaid improving cycle the health condition of the human body will be further improved. The high frequency oscillating chip 3 is a passive element and the entire transmission and amplification are in a passive mode that neither radioactive hazard nor decay of radiation exists for harming a human body. Further the passive mode of picking up and transmitting the human biological micro-wave will concentrate the resonance frequency so as to achieve advantage of a permanent resonance with a human body by a concentrated resonance frequency without hazard from radiation.

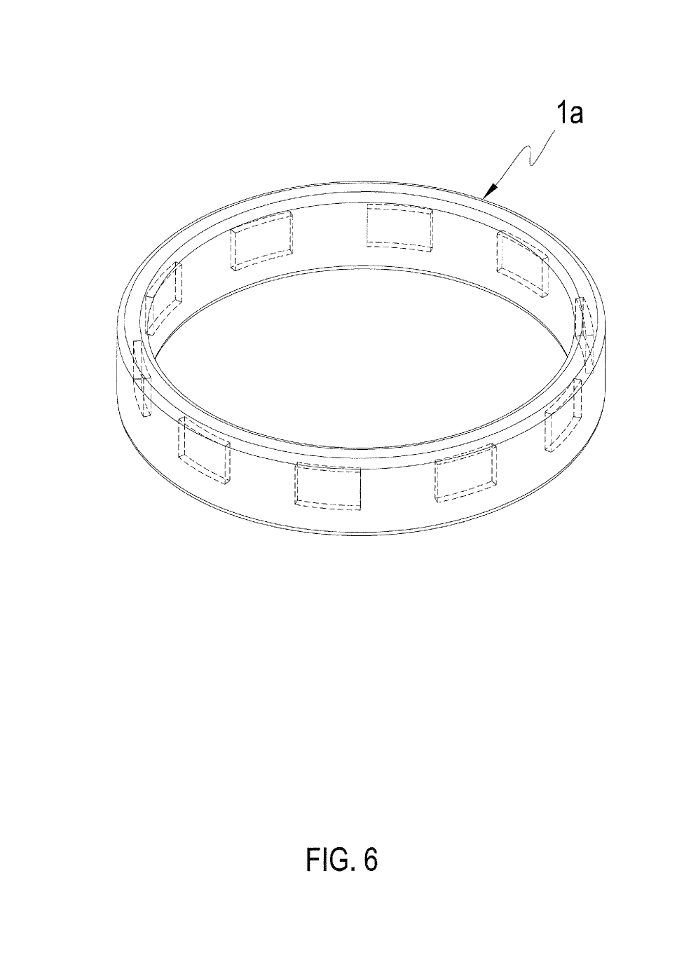

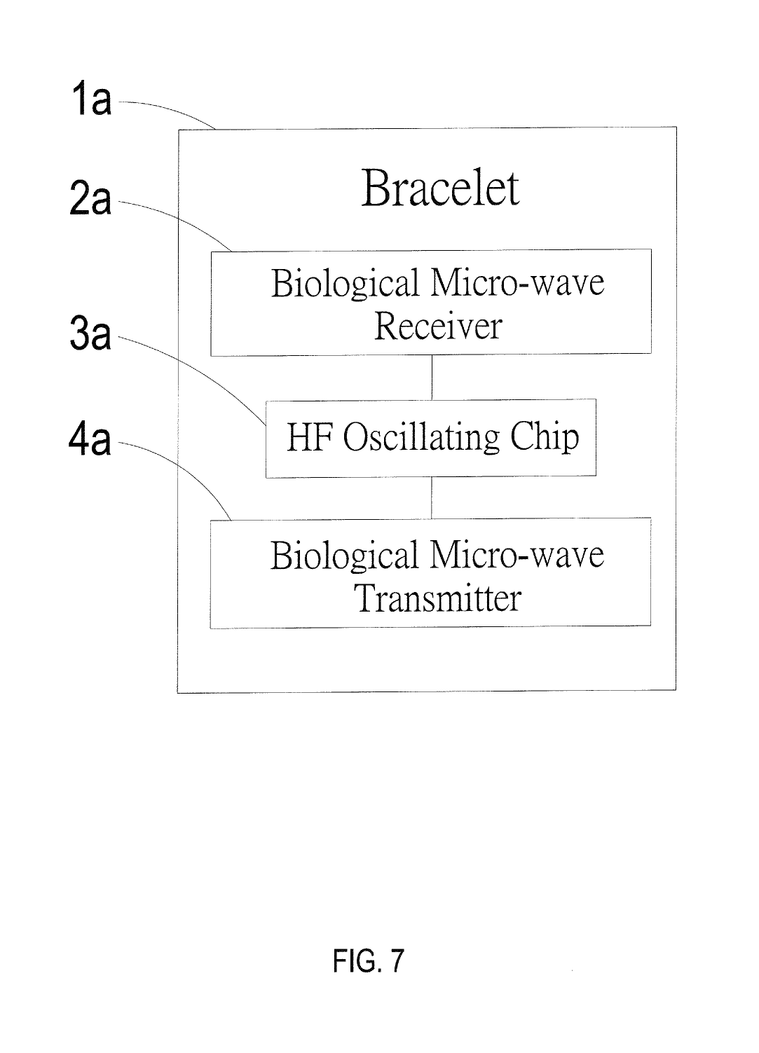

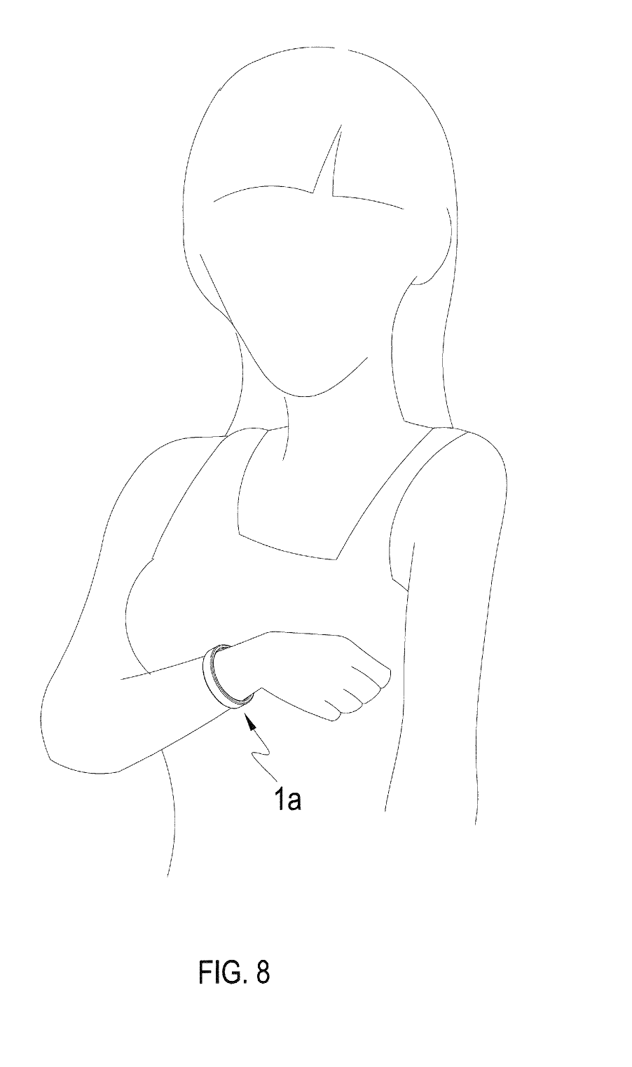

Please refer FIGS. 6 to 8 illustrating a three-dimensional diagram, a structural block diagram and schematic diagram for operation of another embodiment of the present invention. This embodiment consists of:

A bracelet 1a, which can be worn by a human;

At least a biological micro-wave receiver 2a, which is positioned at the bracelet 1a, for picking up a human biological micro-wave emitted from the human body;

A high frequency oscillating chip 3a, which is positioned at the bracelet 1a, for passively receiving a transmission of human biological micro-wave from the biological micro-wave receiver 2a and then amplifying a power of the transmission of human biological micro-wave accordingly; and

A biological micro-wave transmitter 4a, which is positioned at the bracelet 1a, for receiving the power amplified transmission of human biological micro-wave from the high frequency oscillating chip 3a and then transmitting the power amplified transmission of human biological micro-wave to the human body.

Both the biological micro-wave receiver 2a and the biological micro-wave transmitter 4a are made of metal substance and the high frequency oscillating chip 3a consists of a biological signal processing circuit and a memory.

The biological micro-wave receiver 2a, the high frequency oscillating chip 3a and the biological micro-wave transmitter 4a are electrically connected.

In this embodiment a bracelet 1a is provided that multiply sets of the biological micro-wave receivers 2a, the high frequency oscillating chip 3a and the biological micro-wave transmitter 4a are positioned at the bracelet 1a to be worn by a user. The operation and principle of the bracelet 1a is the same as the above-mentioned that they are omitted hereinafter. The bracelet 1a can be substituted by different articles of various forms with the same advantages of a permanent resonance with a human body by a concentrated resonance frequency without hazard from radiation as the above-mentioned.

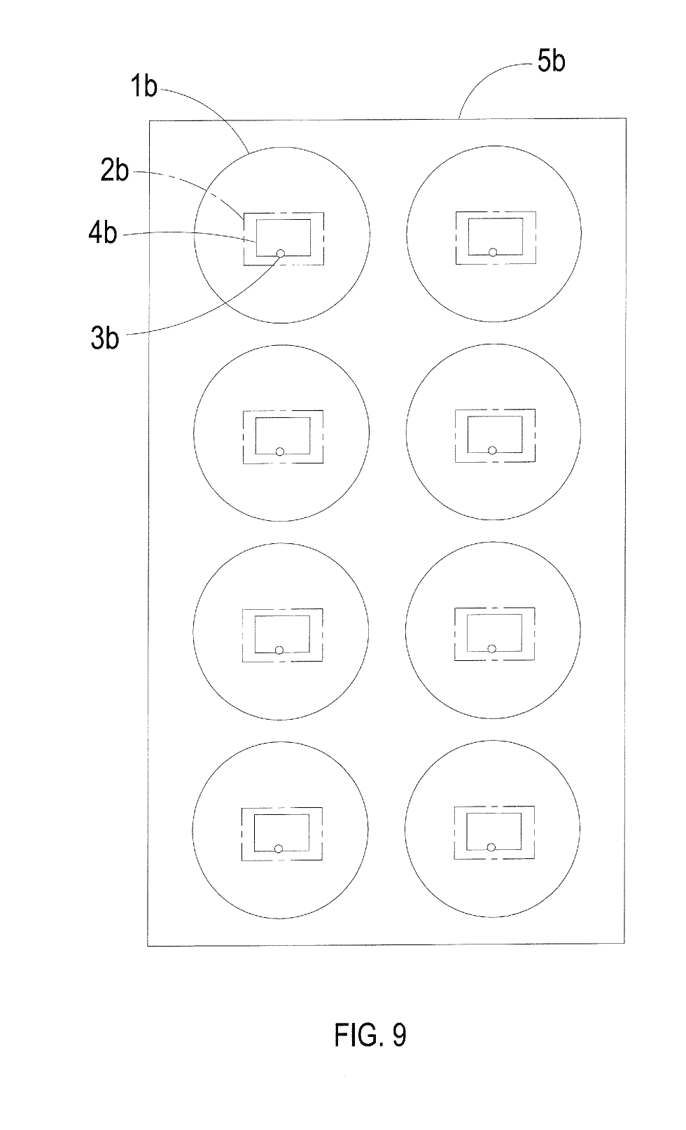

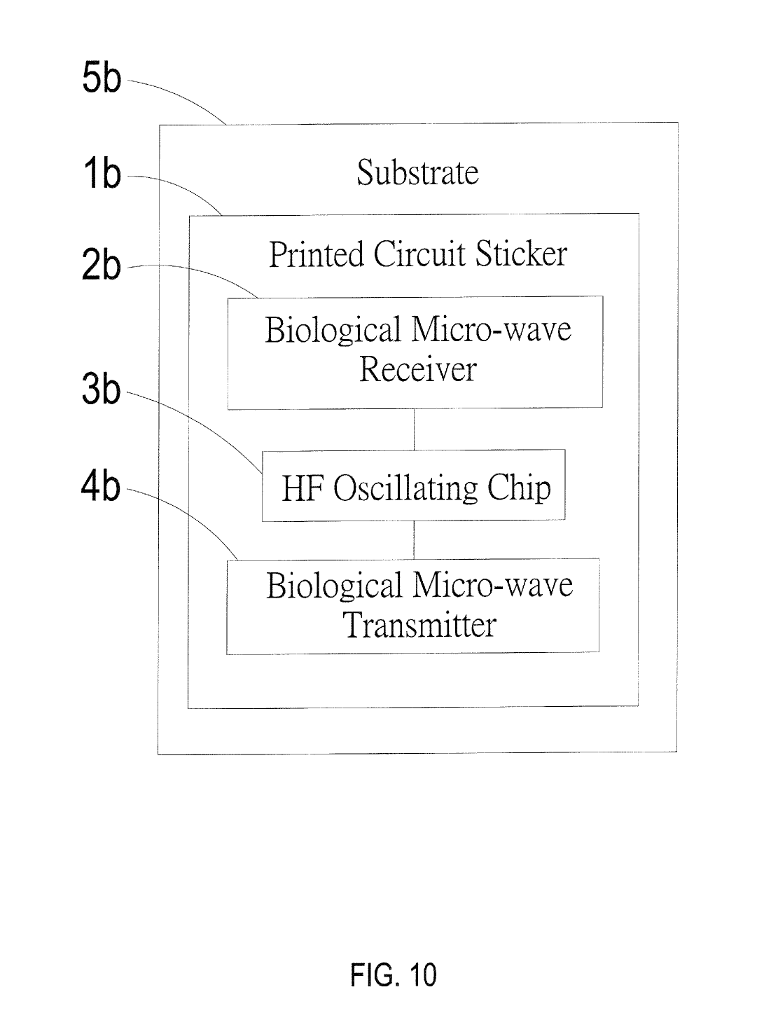

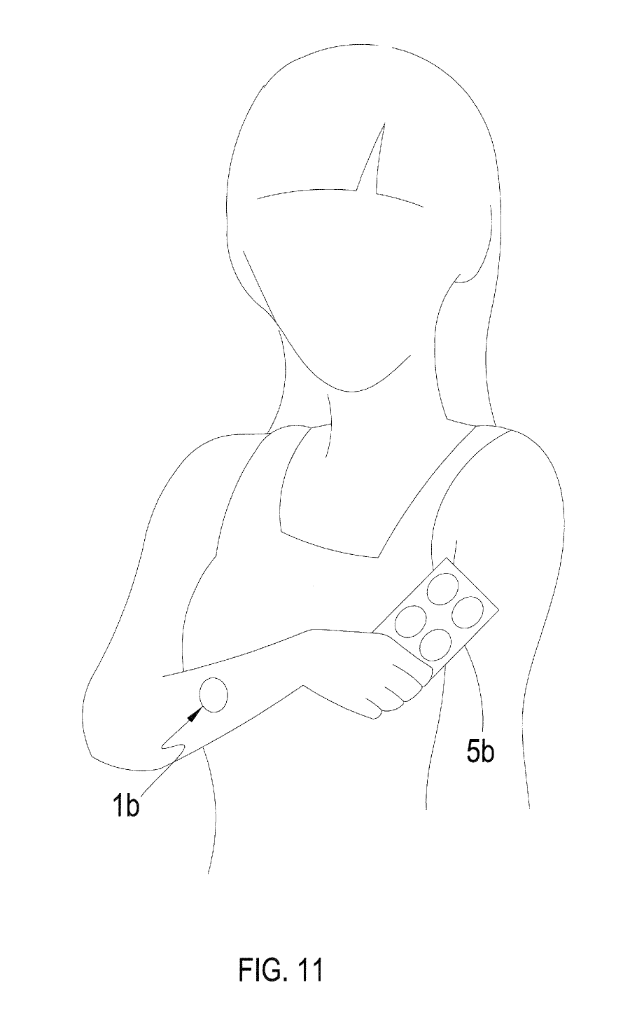

Please refer FIGS. 9 to 11 illustrating a front view diagram, a structural block diagram and a schematic diagram for operation of the other embodiment of the present invention. This embodiment consists of:

A substrate 5b, on which multiply printed circuit stickers 1b are provided, for being placed on an arbitrary area of a human body;

At least a biological micro-wave receiver 2b, which is positioned at the printed circuit stickers 1b, for picking up a human biological micro-wave emitted from the human body;

A high frequency oscillating chip 3b, which is positioned at the printed circuit stickers 1b, for passively receiving a transmission of human biological micro-wave from the biological micro-wave receiver 2b and then amplifying a power level of the transmission of human biological micro-wave accordingly; and

A biological micro-wave transmitter 4b, which is positioned at the printed stickers 1b, for receiving the power amplified transmission of human biological micro-wave from the high frequency oscillating chip 3b and then transmitting the power amplified transmission of human biological micro-wave to the human body.

Both the biological micro-wave receiver 2b and the biological micro-wave transmitter 4b are made of metal substance and the high frequency oscillating chip 3b consists of a biological signal processing circuit and a memory.

The biological micro-wave receiver 2b, the high frequency oscillating chip 3b and the biological micro-wave transmitter 4b are electrically connected.

In this embodiment multiply printed circuit stickers 1b are fabricated on a substrate 5b that a user can take a suitable quantity of the printed circuit stickers 1b from the substrate 5b and place the printed circuit stickers 1 b on an area demanding improvement on health condition. The principle and operation of this embodiment is the same as the above-mentioned that they are omitted hereinafter. The printed circuit stickers 1b can achieve the same advantages of a permanent resonance with a human body by a concentrated resonance frequency without hazard from radiation as the above-mentioned.

The critical technique of the biological resonance chip of the present invention over the prior art is:

1. The biological micro-wave receiver 2 is utilized to enhance the effectiveness of picking up a human biological micro-wave, the high frequency oscillating chip 3 is utilized to amplify a power of the transmission of the human biological micro-wave in receipt and then the biological micro-wave transmitter 4 is utilized to transmit the power amplified transmission of human biological micro-wave to the human body. Therefore the advantages of a permanent resonance with a human body by a concentrated resonance frequency without hazard from radiation are achieved through the aforesaid configuration.
2. The high frequency oscillating chip 3 is utilized to produce high frequency induction shocks with the human biological micro-wave while both the biological micro-wave receiver 2 and the biological micro-wave transmitter 4 are in a rectangle shape, which is of biological micro-wave induction coupling design at a length of 11 mm and a width of 8 mm.

I claim:

1. A biological resonance chip comprising a printed circuit sticker able to be placed on an arbitrary area of a human body;
at least a biological micro-wave receiver positioned on said printed circuit sticker for picking up a human biological micro-wave emitted from the human body;
a high frequency oscillating chip positioned on said printed circuit sticker for passively receiving a transmission of human biological micro-wave and amplifying a power level thereof from said biological micro-wave receiver accordingly, and
at least a biological micro-wave transmitter positioned on said printed circuit sticker for receiving the power amplified transmission of human biological micro-wave from said high frequency oscillating chip and then transmitting the power amplified transmission of human biological micro-wave to the human body.

2. The biological resonance chip of claim 1 wherein both said biological micro-wave receiver and said biological micro-wave transmitter are of metal substance.

3. The biological resonance chip of claim 1 wherein said high frequency oscillating chip comprises a biological signal processing circuit and a memory.

4. The biological resonance chip of claim 1 wherein said printed circuit sticker can be placed on a bracelet.

5. The biological resonance chip of claim 1 wherein said biological micro-wave receiver, said high frequency oscillating chip and said biological micro-wave transmitter are electrically connected.

6. The biological resonance chip of claim 1 wherein said high frequency oscillating chip undergoes a high frequency induction shocks with the transmission of human biological micro-wave and both said biological micro-wave receiver and said biological micro-wave transmitter are in a rectangle shape at a length of 11 mm and a width of 8 mm for induction coupling of human biological micro-wave.
